(19) United States
(12) Patent Application Publication
(10) Pub. No.: US 2026/0258395 A1
(43) Pub. Date: Sep. 3, 2026

(54) Microfluidics-based preparation of cross-linked enzyme aggregates

(71) Applicant: UNIVERZA V LJUUBLJANI, Ljubljana (SI)

(72) Inventors: Tadej Menegatti, Ljubljana (SI); Zan Lavric, Ljubljana (SI); Polona Znidarsic-Plazl, Ljubljana (SI)

(21) Appl. No.: 18/846,832

(22) PCT Filed: Mar. 15, 2023
(86) PCT No.: PCT/EP2023/056602

§ 371 Date: Sep. 13, 2024

(30) Foreign Application Priority Data
Mar. 15, 2022 (LU) LU501664

(51) Int. Cl. C12N 9/96; C12M 3/06; C12N 9/10; C12N 11/087; C12P 13/00

(52) U.S. Cl. CPC C12N 9/96; C12M 23/16; C12N 9/1096; C12N 11/087; C12P 13/001; C12Y 206/01

(57) ABSTRACT

The present invention relates to crosslinked enzyme aggregates (CLEAs) with beneficial properties, and to methods for their preparation. The present invention also pertains to membrane microreactors and microfluidic systems containing such membrane microreactors, which are useful in the preparation and in situ immobilization of CLEAs of the present invention.

### FIELD OF THE INVENTION

The present invention relates to crosslinked enzyme aggregates (CLEAs) with beneficial properties, and to methods for their preparation. The present invention also pertains to membrane microreactors and microfluidic systems containing such membrane microreactors, which are useful in the preparation and in situ immobilization of CLEAs of the present invention.

### BACKGROUND OF THE INVENTION

Safe, environmentally friendly and bio-based production processes are becoming increasingly important if the goals of the circular economy and sustainable development are to be pursued. Located at the roots of bioeconomy, biotechnology increases its market value through novel process design that promotes “greener” production of various value-added molecules. Moreover, its implementation leads to a reduction in waste generated in the process (E-factor), lower energy consumption, operation at ambient pressure and temperature, a reduction in organic solvents, and the use of enzymes instead of ecologically problematic metal-based catalysts (Sheldon & Woodley, 2018; Aguilar et al., 2019).

Despite their sustainability, the use of biocatalysts in industrial production is hindered by the often-perceived low enzyme activity for non-natural substrates and the low operational stability of biocatalysts. This is particularly problematic at high temperatures and high substrate concentrations at industrial scale, which often require the use of organic solvents, leading to either substrate or product inhibition. The application of protein engineering techniques with directed evolution, as well as biocatalyst immobilization, process intensification and integration into downstream processing, can significantly improve the industrial relevance of these green catalysts (Savile et al., 2010; Di Cosimo et al., 2013; Žnidaršič-Plazl, 2021).

A promising technique for biocatalyst immobilization is the formation of cross-linked enzyme aggregates (CLEAs), which are clusters of covalently bound enzymes. Unlike covalently bound enzymes on solid inert supports, CLEAs provide more active sites, resulting in better biocatalyst productivity. The formation of CLEAs typically occurs in two steps: in the first step, enzyme is precipitated (usually with an organic solvent or an inorganic salt), and in the second step, a crosslinking agent is added to react with the amino acid residues and covalently crosslink the precipitated enzyme. Among the organic solvents, ethanol, methanol, acetonitrile, and acetone are most widely used, while glutaraldehyde (GA) is the most typical crosslinking molecule (Sheldon & Woodley, 2018; Guisan et al., 2020).

Batch synthesis of CLEAs results in particles with a diameter of several 100 μm and a heterogenous size distribution. Further use of such particles in continuous systems is challenging due to limited mass transfer, as well as packed bed reactor packing problems leading to the formation of channels and dead zones and thus low reactor performance (Žnidaršič-Plazl, 2021). On the other hand, larger CLEA particles are industrially beneficial as they are more resistant to high shear forces in the stirred tank reactors or large pressure drops in conventional packed bed reactors (Sheldon et al., 2021).

The activity of heterogeneous catalysts depends on their size and porosity. Smaller particles with a large relative surface area allow better diffusion of substrates and products to/from the catalytic site.

The use of microflow systems has recently been discovered as a promising alternative to batch production of CLEAs, where process control is severely limited. The synthesis of cellulase-CLEA particles in microtubes in which nano-cellulase-CLEAs were simultaneously precipitated and crosslinked has been proposed (Nguyen & Yang, 2014). Hollow laccase-CLEAs for dye degradation were prepared in a similar system, which exhibited higher recovered activity compared to non-hollow CLEAs because the active site was more accessible to the substrate (Nguyen et al., 2017). Jannat et al. constructed a similar microfluidic device in which precipitant and crosslinking agent were introduced separately into a tube to simultaneously precipitate and crosslink catalase and produce CLEAs that were then used for H₂O₂ degradation (Jannat & Yang, 2020).

The drawback of the known processes is that the formation of CLEAs cannot be well controlled and require downstream processing such as centrifugation, resulting in CLEAs with low retained enzyme activity. Hence, there remains a need to provide improved methods for the preparation of CLEAs.

### SUMMARY OF THE INVENTION

The present inventors have developed a new microfluidics-based method for the preparation of crosslinked enzyme aggregates (CLEAs). In contrast to previous reports on the preparation of CLEAs in microfluidic systems, in the inventors approach the precipitation and cross-linking are carried out in separate but interconnected parts, which allows optimization of each step and thus better final production of the enzyme.

The present invention thus provides a method for the preparation of cross-linked enzyme aggregates (CLEAs) characterized in that the precipitation and cross-linking are not carried out simultaneously.

The present invention further provides cross-linked enzyme aggregates (CLEAs) obtainable by the method according to the present invention.

The present invention further provides a method for the preparation of a membrane microreactor comprising at least one immobilized enzyme, the method comprises the steps of a) obtaining at least one (such as a plurality of) CLEA according to the present invention, and b) immobilizing the CLEA on the membrane surface of a membrane microreactor.

The present invention further provides a membrane microreactor characterized in that at least one (such as a plurality of) CLEA according to the present invention is immobilized on the membrane surface of the membrane microreactor.

The present invention further provides a microfluidic system comprising a first reaction region, such as a first reaction vessel, a second reaction region, such as a second reaction vessel, which is in fluidic connection with the first reaction region, and a membrane microreactor, which is in fluidic connection with the second reaction region.

The present invention can be further summarized by the following items.
1. A method for the preparation of cross-linked enzyme aggregates (CLEAs), the method comprising the steps:
   a) precipitation of at least one enzyme, optionally together with its required co-factor(s), in the presence of a precipitating agent; and
   b) reacting the precipitated enzyme(s) with a crosslinking agent to obtain said CLEAs;
   wherein steps a) and b) are not carried out simultaneously.
2. The method according to item 1, wherein step a) comprises providing a buffered solution comprising a suitable amount of the enzyme, such as in the range from about 1 mg/ml to about 10 mg/mL, and adding the precipitating agent.
3. The method according to item 1 or 2, wherein the precipitating agent is selected from the group consisiting of ethanol, methanol, propanol, such as 1-propanol or 2-propanol, butanol, such as tert-butanol, acetonitrile, acetone, ethyl lactate, dimethoxyethane (DME), dimethylformamide (DMF), dimethyl sulfoxide (DMSO) and polyethylene glycol (PEG).
4. The method according to any one of items 1 to 3, wherein the precipitating agent is acetone.
5. The method according to any one of items 1 to 4, wherein the precipitating agent is added at a concentration ranging from about 5 vol. % to about 100 vol. %, about 50 vol. %.
6. The method according to any one of items 1 to 5, wherein the crosslinking agent is selected from the group consisting of glutaraldehyde, N-methylenebisacrylamide, bismaleimide, dextran, p-benzoquinone, glucoamylase and chitosan.
7. The method according to any one of items 1 to 6, wherein the crosslinking agent is glutaraldehyde.
8. The method according to any one of items 1 to 7, wherein step b) comprises adding a buffered solution comprising the crosslinking agent, preferably at a concentration ranging from about 1 mM to about 100 mM, such as from about 3 mM to about 50 mM.
9. The method according to any one of items 1 to 8, wherein the method is carried out in a microfluidic system.
10. The method according to item 9, wherein the microfluidic system is a microfluidic device.
11. The method according to item 9 or 10, wherein the microfluidic system comprises a first reaction region, such as a first reaction vessel or chamber, and a second reaction region, such as a second reaction vessel or chamber, both reaction regions being in fluidic connection with each other.
12. The method according to item 11, wherein step a) is carried out in the first reaction region and step b) is carried out in the second reaction region.
13. The method according to item 12, wherein the first reaction region comprises at least one inlet which is in fluidic connection with at least one injection means.
14. The method according to item 13, wherein the at least one injection means is in fluidic connection with the at least one inlet of the first reaction region via a first micromixer.
15. The method according to item 14, wherein the first micromixer is in fluidic connection with a first injection means and a second injection means, such as a first microfluidic pressure pump and a second microfluidic pressure pump.
16. The method according to item 15, wherein the solution comprising the at least one enzyme is added to the micromixer via the first injection means and the precipitating agent is added to the micromixer via the second injection means.
17. The method according to any one of items 11 to 16, wherein the solution comprising the at least one enzyme and the precipitating agent are added simultaneously to the first reaction region.
18. The method according to item 16 or 17, wherein the solution comprising the at least one enzyme and the precipitating agent are added at equal flow rates ranging from about 25 μL/min to about 100 μL/min, such as at equal flow rates of about 50 μL/min.
19. The method according to any one of items 11 to 16, wherein the second reaction region comprises at least one inlet which is in fluidic connection with at least one (such as a third) injection means, such as a (third) microfluidic pressure pump.
20. The method according to item 19, wherein the at least one injection means is in fluidic connection with the at least one inlet of the second reaction region via a second micromixer.
21. The method according to item 20, wherein the second micromixer is located between the first reaction region and second reaction region.
22. The method according to any one of items 11 to 21, comprising transferring the precipitated enzyme(s) obtained in step a) from the first reaction region to the second reaction region, and adding the buffered solution comprising the crosslinking agent to the second reaction region to initiate step b).
23. The method according to any one of items 19 to 22, wherein the solution comprising the crosslinking agent is added to the second reaction region via the third injection means.
24. The method according to item 22 or 23, wherein the crosslinking agent is added to the second reaction region at a flow rate ranging from about 50 μL/min to about 200 μL/min, such as at a flow rate of about 100 L/min.
25. The method according to any one of items 11 to 24, wherein the second reaction region comprises at least one outlet.
26. The method according to any one of items 10 to 24, wherein the microfluidic system comprises a collection region, such as a collection vessel or chamber, which is in fluidic connection with the second reaction region.
27. The method according to item 26, wherein the cross-linked enzyme aggregates obtained in step b) are collected in said collection region.
28. The method according to any one of items 1 to 29, which is carried out in a microfluidic system according to any one of items 55 to 69.
29. The method according to any one of items 1 to 28, wherein the at least one enzyme is selected from the group consisting of transferases such as amine transaminases, oxidoreductases, such as oxidases, peroxidases, laccases, ketoreductases, imine reductases, and carbamoylases, dehydrogenases, hydrolases such as esterases, lactamases such as beta-lactameses, proteases, cellulases, lipases, aminopeptidases, nitrilases, xylanases, glycosylases, amidases, lyases, such as hydroxynitrile lyases, and aldolases.
30. The method according to any one of items 1 to 29, wherein the enzyme is a transaminase.
31. The method according to item 30, wherein the enzyme is an amine transaminase.
32. Cross-linked enzyme aggregate (CLEA) obtainable by the method according to any one of items 1 to 31.
33. The CLEA according to item 32, having a mean particle size distribution (d50) of about 200 nm or below.
34. The CLEA according to any one of items 32 to 33, having a particle size distribution (d50) ranging from about 30 nm to about 200 nm.
35. The CLEA according to any one of items 32 to 34, having a particle size distribution (d50) ranging from about 30 nm to about 100 nm.
36. The CLEA according to any one of items 32 to 35, having a polydespersity index of about 0.27 or below.
37. The CLEA according to any one of items 32 to 36, having a polydispersity index ranging from about 0.19 to about 0.27.
38. A method for the preparation of a solid support comprising at least one immobilized enzyme, the method comprises the steps of a) obtaining at least one (such as a plurality of) CLEA according to the method according to items 1 to 31, and b) immobilizing the CLEA(s) on or within a solid support.
39. The method according to item 38, wherein the solid support is selected from the group consisting of membranes, porous or non-porous beads, porous or non-porous particles, hollow fibers, monolithic columns, microchannels and microchambers.
40. The method according to item 38 or 39, wherein the solid support is a membrane, such as an ultrafiltration membrane.
41. A method for the preparation of a microfluidic device comprising at least one immobilized enzyme, the method comprises the steps of a) obtaining at least one (such as a plurality of) CLEA according to the method according to any one of items 1 to 31, and b) immobilizing the CLEA(s) on or within a surface of a microfluidic device.
42. The method according to item 41, wherein the microfluidic device is selected from the group consisting of membrane microreactors, hollow fiber bioreactors, packed bed reactors, monolithic columns, microchannels or microchambers, preferably membrane microreactor is a membrane microreactor.
43. A method for the preparation of a membrane microreactor comprising at least one immobilized enzyme, the method comprises the steps of a) obtaining at least one (such as a plurality of) CLEA according to the method according to any one of items 1 to 31, and b) immobilizing the CLEA(s) on the membrane surface of a membrane microreactor.
44. The method according to item 43, wherein the membrane microreactor comprises an ultrafiltration membrane.
45. The method according to item 44, wherein the ultrafiltration membrane has a molecular weight cut off (MWCO) ranging from about 100 kDA to about 300 kDA.
46. The method according to 44 or 45, wherein the membrane microreactor comprises two poly(methylmethacrylate) (PMMA) plates with inserted polytetrafluoroethylene (PTFE) spacers and said ultrafiltration membrane.
47. The method according to any one of items 38 to 46, further comprising c) washing the immobilized CLEA particles.
48. A solid support characterized in that at least one (such as a plurality of) CLEA according to any one of items 32 to 37 is (are) immobilized on or within.
49. The solid support according to item 48, which is selected from the group consisting of membranes, porous or non-porous beads, porous or non-porous particles, hollow fibers, monolithic columns, microchannels and microchambers.
50. The solid support according to item 48 or 49, which is a membrane such as such as an ultrafiltration membrane.
51. A membrane microreactor characterized in that at least one (such as a plurality of) CLEA according to any one of items 32 to 37 is (are) immobilized on the membrane surface of the membrane microreactor.
52. The membrane microreactor according to item 51, wherein the membrane microreactor comprises an ultrafiltration membrane to which said CLEA particels are immobilized.
53. The method according to item 52, wherein the ultrafiltration membrane has a molecular weight cut off (MWCO) ranging from about 100 kDA to about 300 kDA.
54. The method according to 52 or 53, wherein the membrane microreactor comprises two poly(methylmethacrylate) (PMMA) plates with inserted polytetrafluoroethylene (PTFE) spacers and said ultrafiltration membrane.
55. A microfluidic system comprising a first reaction region, such as a first reaction vessel, a second reaction region, such as a second reaction vessel, which is in fluidic connection with the first reaction region, and an immobilization region, which is in fluidic connection with the second reaction region.
56. The microfluidic system according to item 55, wherein the first reaction region comprises at least one inlet which is in fluidic connection with at least one injection means.
57. The microfluidic system according to item 56, wherein the at least one injection means is in fluidic connection with the at least one inlet of the first reaction region via a first micromixer.
58. The microfluidic system according to item 57, wherein the first micromixer is in fluidic connection with a first injection means and a second injection means, such as a first microfluidic pressure pump and a second microfluidic pressure pump.
59. The microfluidic system according to any one of items 55 to 58, wherein the second reaction region comprises at least one inlet which is in fluidic connection with at least one (such as a third) injection means, such as a (third) microfluidic pressure pump.
60. The microfluidic system according to item 59, wherein the at least one injection means is in fluidic connection with the at least one inlet of the second reaction region via a second micromixer.
61. The microfluidic system according to item 60, wherein the second micromixer is located between the first reaction region and second reaction region.
62. The microfluidic system according to any one of items 55 to 61, wherein the second reaction region comprises at least one outlet.
63. The microfluidic system according to item 62, wherein the membrane microreactor is in fluidic connection with the second reaction region via said outlet.
64. The microfluidic system according to any one of items 55 to 63, wherein said immobilization region is selected from the group consisting of membrane microreactors, hollow fiber bioreactors, packed bed reactors, monolithic columns, microchannels and microchambers, preferably said immobilization region is a membrane microreactor.
65. The microfluidic system according to item 64, wherein the membrane microreactor comprises an ultrafiltration membrane.
66. The microfluidic system according to item 65, wherein the ultrafiltration membrane has a molecular weight cut off (MWCO) ranging from about 100 kDA to about 300 kDA.
67. The microfluidic system according to 64 or 65, wherein the membrane microreactor comprises two poly(methylmethacrylate) (PMMA) plates with inserted polytetrafluoroethylene (PTFE) spacers and said ultrafiltration membrane.
68. The microfluidic system according to any one of items 55 to 67, wherein at least one (such as a plurality of) cross-linked enzyme aggregate (CLEA) is (are) immobilized on or within the immobilization region, such as the membrane surface of a membrane microreactor.
69. The microfluidic system according to any one of items 55 to 68, characterized in that at least one (such as a plurality of) CLEA according to any one of items 32 to 37 is (are) immobilized on or within the immobilization region, such as the membrane surface of a membrane microreactor.
70. A process for producing a chiral amine comprising the step of catalyzing a transamination reaction using an amine transaminase cross-linked enzyme aggregate (ATA-CLEA) on a ketone compound and an amino donor, wherein the ATA-CLEA is a ATA-CLEA obtainable by the method according to item 31.

### BRIEF DESCRIPTION OF THE DRAWINGS

FIG. 1: Schematic presentation of a microfluidic system for CLEA particles preparation and in situ immobilization in a membrane microreactor.

FIG. 2: Schematic presentation of a membrane microreactor. a) presentation of the membrane microreactor components, b) simultaneous CLEAs immobilization and cleaning, c) continuous biotransformation with CLEAs inside a membrane microreactor

FIG. 3: DLS measurement of particles obtained at various enzyme and acetone concentrations in the microfluidic set-up with 3 inlets. For each sample, 10 measurements were performed, each presented with a different color. Total flow rate in the second tube was 200 μl min-1 and GA concentration was 12.5 mM, while enzyme (Cₑ) and acetone concentrations were as follows: a) Cₑ 2.5 mg mL⁻¹, acetone 50 vol. %, b) Cₑ 1 mg mL⁻¹, acetone 50 vol. %, c) Cₑ=1 mg mL⁻¹, acetone 12.5 vol. %, d) Cₑ 1 mg mL⁻¹, acetone 3.125 vol. %.

FIG. 4: The effect of acetone and GA concentration on the recovered activity of ATA-CLEAs obtained in a microfluidic set-up at 1 mg mL⁻¹ enzyme concentration. Red dots (·) represent the experimental points.

FIG. 5: Gross yield of ACP dependence on the residence time in the membrane microreactor with ATA-CLEA particles and nonaggregated ATA-v1. Experiment was carried out at 50° C. and with 1.86 mg mL⁻¹ enzyme concentration in the microreactor.

FIG. 6: Time dependence of a 100-kDa membrane microreactor productivity relative to initial productivity, which was 0.36 and 0.29 U mg-1 for ATA-CLEA and nonaggregated enzyme enzyme, respectively. Experiments were carried out at 50° C., at flow rate of 5 μL min-1 and at 1.86 mg mL-1 enzyme concentration in the microreactor.

### DETAILED DESCRIPTION OF THE INVENTION

As mentioned above, the present inventors have developed a new microfluidic-based method for the preparation of crosslinked enzyme aggregates (CLEAs). In contrast to previous reports on the preparation of CLEAs in microfluidic systems, in the inventors approach the precipitation and cross-linking are carried out in separate but interconnected parts, which allows optimization of each step and thus better final production of the enzyme.

In the underlying experiments performed by the present inventors, amine transaminase (ATA) has been used a non-limiting model enzyme for the preparation of cross-linked enzyme aggregates. Amine transaminases (ATAs), which transfer an amino group of the donor molecule to a ketone or aldehyde, are highly enantioselective enzymes that can synthesize optically pure amines (Malik et al., 2012, Kelly et al., 2018). They require a cofactor, namely pyridoxal-5′phosphate (PLP), which is responsible for binding the amine moiety in the transamination step. Most ATAs have a high affinity for the cofactor, which plays a key role in stabilizing the enzyme (Börner et al., 2017).

After optimizing the concentrations of acetone and glutaraldehyde for precipitation and crosslinking, respectively, ATA-CLEAs prepared in a microfluidic device exhibited 87.1% recovered activities, which is 2.4-fold higher than the values previously reported for ATA-CLEAs. The prepared CLEA particles with an average particle radius of 37.13±0.38 nm and polydispersity index of 0.19 showed also higher activity than the free enzyme at 70° C. Further integration of a membrane microreactor into the system for ATA-CLEA production enabled in situ immobilization of the enzyme preparation and the continuous transamination of a model compound. The immobilization yield for ATA-CLEA in the microreactor with 100 kDa molecular weight cut off membrane was 100% and the immobilization efficiency achieved was 60.5%, which was more than 20% better than results obtained for nonaggregated enzyme immobilization on the same membrane.

Besides, ATA-CLEA particles showed much better operational stability than nonaggregated enzyme in a membrane microreactor, as productivity remained constant during the 6 days of continuous operation.

Based on the present inventors' research the present invention has been completed.

Thus, the present invention provides in a first aspect a method for the preparation of cross-linked enzyme aggregates (CLEAs), the method comprising the steps:
a) precipitation of at least one enzyme, optionally together with its required co-factor(s), in the presence of an precipitating agent; and
b) reacting the precipitated enzyme(s) with a crosslinking agent to obtain said CLEAs;
wherein steps a) and b) are not carried out simultaneously.

The method of the present invention thus involces two basic reactions. First, the enzyme(s) in solution is precipitated using a precipitating agent to form enzyme aggregates. Second, the enzyme aggregates are then submitted to crosslinking by a crosslinking agent to fix the structure of the enzyme aggregates.

The method of the present invention may involve the processing of one enzyme and, where applicable and desired, its required co-factor(s). However, it is also contemplate that a plurality of different enzymes (i.e. at least two enzymes), optionally together with their required co-factor(s), are precipitated and cross-linked, resulting in so-called “Combi-CLEA”. Co-immobilization of two or more enzymes in Combi-CLEAs enables the cost-effective use of multiple enzymes in biocatalytic cascade processes. To this end, a solution comprise two or more enzymes and, where applicable and desired, their required co-factor(s) is subjected to precipitation. Alternatively, separate precipitation reaction for each of the enzymes may be carried out, in a sequential order, and the precipitated enzymes may then be combined for the subsequent cross-linking step. Optionally, magnetizable (nano-)particles, such as particles of a zerovalent metal selected from the group of iron, nickel, cobalt and alloys thereof, may be added during the cross-linking step to form magnetic CLEAs, which allows separation of the aggregated enzyme from other solids.

Generally, the precipitation step a) may be performed at any suitable temperature which allows the precipitation of the enzyme(s) of interest, but normally may be performed at a temperature in the range from about 0° C. to about 40° C., such as in the range from about 20° C. to about 30° C. According to some embodiments, the precipitation step a) is performed at a temperature in the range from about 0° C. to about 30° C. According to some embodiments, precipitation step a) is performed at a temperature in the range from about 4° C. to about 30° C., such as from about 4° C. to about 25° C. According to some embodiments, precipitation step a) is performed at a temperature in the range from about 10° C. to about 30° C., such as from about 15° C. to about 25° C. According to some embodiments, precipitation step a) is performed at a temperature in the range from about 0° C. to about 10° C., such as from about 0° C. to about 4° C.

The precipitation is preferably performed in a buffered environment with a pH suitable for the enzyme(s) of interest. In practice, the pH normally ranges from about 4 to about 11, preferably from about 5 to about 9, such as from about 6 to about 8.

Thus, according to some embodiments, step a) comprises providing a buffered solution comprising a suitable amount of the enzyme(s), optionally together with their required co-factor(s), and adding the precipitating agent.

The at least one enzyme to be used in the present invention for the preparation of cross-linked enzyme aggregates may be any desired enzyme. Non-limiting examples are transferases such as amine transaminases, oxidoreductases such as oxidases, peroxidases, laccases, ketoreductases, imine reductases, and carbamoylases, dehydrogenases, hydrolases such as esterases, lactamases such as beta-lactameses, proteases, cellulases, lipases, aminopeptidases, nitrilases, xylanases, glycosylases, amidases, lyases, such as hydroxynitrile lyases, and aldolases. According to some embodiments, the enzyme is a transaminase, and preferably is an amine transaminase. According to some embodiments, the enzyme is a laccase.

The optimal amount of the enzymes to be used is largely dependend on the specific enzyme chosen, and can easily be determined by the skilled person. A suitable amount of the enzyme may, for example, range from about 1 mg/ml to about 100 mg/mL, such as from about 1 mg/ml to about 50 mg/ml, from about 1 mg/ml to about 10 mg/ml, from about 2 mg/ml to about 10 mg/ml, from about 2 to about 5 mg/ml, or from about 3 mg/ml to about 5 mg/mL. The final concentration of the enzyme upon addition of the precipitating agent will depend on the flow rate ratio of enzyme to precipitating agent solutions used, which may be in the range of 1:0.5 to 1:10, such as from 1:1 to 1:5. Preferably, the flow rate ratio of enzyme to precipitating agent solutions used is 1:1.

Suitable precipitating agents that can be used in the method of the present invention are in principle all water soluble precipitating agents that are used in the art of precipitation of biomolecules, for instance organic solvents and (bio) polymers. Some generally applicable and well known precipitating agents are organic solvents like methanol, ethanol, propanol, isopropanol, butanol, and tert-butylalcohol, acetone, acetonitrile, ethyl lactate, dimethoxyethane (DME), dimethylformamide (DMF), dimethyl sulfoxide (DMSO) or any of the poly(ethylene glycol) (PEG) series.

Thus, according some embodiments, the precipitating agent is an organic solvent.

According to some embodiments, the precipitating agent is selected from the group consisiting of ethanol, methanol, propanol, such as 1-propanol or 2-propanol, butanol, tert-butylalcohol, acetonitrile, acetone, ethyl lactate, dimethoxyethane (DME), dimethylformamide (DMF), dimethyl sulfoxide (DMSO) and polyethylene glycol (PEG), such as a polyethylene glycol with a molecular weight preferably between 3000 and 16000.

According to some embodiments, the precipitating agent is acetone.

The optimal amount of precipitating agent to be used is largely dependend on the specific precipitating agent chosen, and can easily be determined by the skilled person. Generally, the amount of precipitating agent added in step a) may range from about 5 vol. % to about 100 vol. %, such as from about 20 vol. % to about 100 vol. %, from about 20 vol. % to about 90 vol. %, from about 20 vol. % to about 50 vol. %. For instance, the optimal amount of acetone to be used ranges from 20 vol. % to 50 vol. %. The final concentration of the precipitating agent will depend on the flow rate ratio of enzyme to precipitating agent solutions used, as mentioned above. For example, if the flow rate ratio is 1:1, the final concentration of the precipitating agent may be in the range of about 2.5 vol. % to about 50 vol. %.

Suitable crosslinking agents to be used are in principle all agents that can be used in the crosslinking of enzymes, such as, but not limited to, glutaraldehyde, N-methylenebisacrylamide, bismaleimide and, dextran, p-benzoquinone, glucoamylase and chitosan. One preferred crosslinking agent is glutaraldehyde.

Thus, according to some embodiments, the crosslinking agent is selected from the group consisting of glutaraldehyde, N-methylenebisacrylamide, bismaleimide and, dextran, p-benzoquinone, glucoamylase and chitosan.

According to some embodiments, the crosslinking agent is glutaraldehyde.

The optimal amount of crosslinking agent to be used is largely dependend on the specific crosslinking agent chosen, and can easily be determined by the skilled person. Generally, the amount of crosslinking agent added in step b) may range from about 1 mM to about 100 mM, such as from about 3 mM to about 50 mM. For instance, the optimal amount of glutaraldehyde to be used ranges from about 3 mM to about 20 mM, such as from about 5 mM to 10 mM. The final concentration of the crosslinking agent in the crosslinking reaction will depend on the flow rate ratio of precipitated enzyme to crosslinking agent solutions used, which may be in the range of 1:0.5 to 1:10, such as from 1:1 to 1:5. Preferably, the flow rate ratio of precipitated enzyme to crosslinking agent solutions used is 1:1.

Crosslinking step b) may be performed at any suitable temperature, which allows the crosslinking of the enzyme(s) in question, but normally may be performed at a temperature in the range from about 0° C. to about 40° C., such as in the range from about 20° C. to about 30° C. According to some embodiments, crosslinking step b) is performed at a temperature in the range from about 0° C. to about 30° C. According to some embodiments, the crosslinking step b) is performed at a temperature in the range from about 4° C. to about 30° C., such as from about 4° C. to about 25° C. According to some embodiments, crosslinking step b) is performed at a temperature in the range from about 10° C. to about 30° C., such as from about 15° C. to about 25° C. According to some embodiments, crosslinking step b) is performed at a temperature in the range from about 0° C. to about 10° C., such as from about 0° C. to about 4° C. According to some embodiments, crosslinking step b) is performed at the same temperature as precipitation step a).

The crosslinking is preferably performed in a buffered environment with a pH suitable for the enzyme(s) of interest. In practice the pH normally ranges from about 4 to about 11, preferably from about 5 to about 9, such as from about 6 to about 8.

Thus, according to some embodiments, step b) comprises adding a buffered solution comprising the crosslinking agent, preferably at a concentration ranging from about 1 mM to about 100 mM, such as from about 3 mM to about 50 mM.

According to some embodiments, step b) comprises adding a buffered solution comprising the crosslinking agent at a concentration ranging from about 1 mM to about 20 mM, such as from about 3 mM to about 20 mM.

According to some embodiments, step b) comprises adding a buffered solution comprising the crosslinking agent at a concentration ranging from about 1 mM to about 10 mM, such as from about 5 mM to about 10 mM.

Optionally, the buffered solution may further comprise magnetic (nano-)particles, such as particles of a zerovalent metal selected from the group of iron, nickel, cobalt and alloys thereof. Alternatively, the magnetic (nano-)particles may be added separately to the crosslinking reaction.

Suitable, the method of the present invention is carried out in a microfluidic system.

Generally, a microfludic system used in accordance with the present invention is a system that can process small quantities of fluids. Suitably, the microfluidic system is configured to carry out the method of the present invention and comprises, as a minimum, a first reaction region, such as a first reaction vessel or chamber, and a second reaction region, such as a second reaction vessel or chamber, which are in fluidic connection with each other. The microfluidic system may be a microfluidic device, which can be newly constructed or may be a commercially available microfluidic device.

Microfluidic devices may be made from polymers, glass, silicon, metal, or other materials, and can be of any suitable type such as a microfluidic chip comprising one or more microchannels and optionally one or more reaction chambers. Non-limiting examples of microfluidic chips useful according to the present invention are lab-on-a-chip (LOC) devises. Alternatively, continuous flow microfluidic channels can be used, wherein fluids are moved within closed channels having characteristic dimensions below about 1 mm, such as capillaries, tubes or plates.

The microfluidic system may have multiple inlets through which the reactants are injected into the system. Suitable, each inlet may be in fluidic connection with at least one injection means, such as a microfluidic pressure pump (e.g., syringe pump, peristaltic pump or piezoelectric pump), either directly or indirectly via one or more micromixers, such as a T- or Y-micromixer(s). Preferably, the injection means is a syringe pump. Suitably, the microfluidic pressure pump can provide for a pressure in the system in the range from 0.1 to 700 bar.

Thus, according to some embodiments, the first reaction region comprises at least one inlet which is in fluidic connection with at least one injection means, such as at least one microfluidic pressure pump, such as at least one syringe pump.

According to some embodiments, the at least one injection means is in fluidic connection with the at least one inlet of the first reaction region via a first micromixer.

According to some embodiments, the first micromixer is in fluidic connection with a first injection means and a second injection means, such as a first microfluidic pressure pump and a second microfluidic pressure pump, such as a first syringe pump and a second syringe pump.

When a microfluidic system is used, step a) is carried out in the first reaction region and step b) is carried out in the second reaction region. Accordingly, in step a) the solution comprising the at least one enzyme and the precipitating agent are each added, preferably simultaneously, to the first reaction region.

Thus, according to some embodiments, the solution comprising the at least one enzyme and the precipitating agent are added simultaneously to the first reaction region.

According to some embodiments, the solution comprising the at least one enzyme is added to the micromixer via the first injection means and the precipitating agent is added to the micromixer via the second injection means.

The solution comprising the at least one enzyme and the precipitating agent may be added at equal flow rates ranging, e.g., from about 25 μL/min to about 200 μL/min, such as from about 25 μL/min to about 100 μL/min, such as at equal flow rates of about 50 μL/min. As will be appreciated by the skilled person, the eventual flow rate will depend on the Reynolds number values in the flow. For example, in laminar flow the Reynolds number values may be in the range from about 1 to about 12. Once both in-flows get in contact, the at least one enzyme and the precipitating agent get mixed and precipitation of the at least one enzymes occurs.

The precipitated enzyme(s) will then be transferred (e.g., flows) into the second reaction region where it is (they are) subjected to crosslinking by the addition of the crosslinking agent. To this end, the second reaction region comprises at least one inlet which is in fluidic connection with at least one (such as a third) injection means, such as a (third) microfluidic pressure pump, such as a (third) syringe pump, either directly or indirectly via a (second) micromixer, such as a T- or Y-micromixer. The (second) micromixer may be located between the first reaction region and second reaction region. Once the precipitated enzyme(s) gets in contact with the in-flow of the crosslinking agent, the precipitated enzyme(s) and the crosslinking agent get mixed and crosslinking occurs.

The solution comprising the crosslinking agent may be added to the second reaction region at a flow rate ranging from about 50 μL/min to about 400 L/min, such as from about 50 μL/min to about 200 μL/min, such as at a flow rate of about 100 μL/min. As will be appreciated by the skilled person, the eventual flow rate will depend on the Reynolds number values in the flow. For example, in laminar flow the Reynolds number values may be in the range from about 2 to about 24.

For downstream applications, such as the collection of the cross-linked enzyme aggregates after completion of the crosslinking reaction, the second reaction region may comprise at least one outlet. The microfluidic system may thus comprises a collection region, such as a collection vessel or chamber, which is in fluidic connection with the second reaction region via said outlet. Hence, the method may comprise collecting the cross-linked enzyme aggregates obtained in step b) in said collection region.

Alternatively, as will be detailed below, the second reaction region may be in fluidic connection with an immobilization region, via said outlet (or any other outlet there may be) for in situ immobilization of the CLEAs. The immobilization region may be a membrane microreactor, a hollow fiber bioreactor, a packed bed reactor, a monolithic column, a microchannel or a microchamber. The microchannel may be a microchannel chip, a microchannel plate, a microchannel tube or a capillary. Preferably, the immobilization region is a membrane microreactor.

According to some embodiments, the method of the present invention is carried out in a microfluidic system according to the present invention (as will be described in more detail below).

The present invention provides in a further aspect a cross-linked enzyme aggregate (CLEA) obtainable by the production method of the present invention.

As mentioned above, the cross-linked enzyme aggregate(s) obtainable by the present invention have beneficial properties. More specifically, the CLEAs have been shown to retain higher activities, have higher size uniformity and exhibit better thermal stability compared to CLEAs obtained with the prior art method.

The CLEA/s of the present invention may be (further) characterized in that it/they has/have a mean particle size distribution (d50) of about 200 nm or below, such as about 150 nm or below. According to some embodiments, the CLEA/s of the present invention has/have a particle size distribution of about 120 nm or below. According to some embodiments, the CLEA/s of the present invention has/have a particle size distribution of about 100 nm or below. According to some embodiments, the CLEA/s of the present invention has/have a particle size distribution (d50) of about 85 nm or below, such as about 82 nm. The CLEA/s of the present invention may have a particle size distribution (d50) ranging from about 30 nm to about 200 nm, such as from about 30 nm to about 150 nm. According to some embodiments, the CLEA/s of the present invention has/have a particle size distribution (d50) ranging from about 50 nm to about 150 nm. According to some embodiments, the CLEA/s of the present invention has/have a particle size distribution (d50) ranging from about 50 nm to about 120 nm. According to some embodiments, the CLEA/s of the present invention has/have a particle size distribution (d50) ranging from about 50 nm to about 100 nm. According to some embodiments, the CLEA/s of the present invention has/have a particle size distribution (d50) ranging from about 70 nm to about 100 nm. According to some embodiments, the CLEA/s of the present invention has/have a particle size distribution (d50) ranging from about 80 nm to about 85 nm.

The CLEA/s of the present invention may be (further) characterized in that it/they has/have a polydispersity index of about 0.27 or below. The CLEA/s of the present invention may have a polydispersity index ranging from about 0.19 to about 0.27.

Particle size distribution can be measured using a Litesizer™ 500 particle size analyzer (Anton-Paar, Graz, Austria) with dynamic light scattering (DLS) being applied. The wavelength of the laser is set to 658 nm and power of 40 mW. A standard operating procedure is set for measuring CLEA in water at 20° C. Each sample is measured 10 times for 30 s. The average particle radius is calculated as an average value of the 10 measurements using the software provided by the manufacturer (Anton-Paar, Graz, Austria) and is reported with standard deviation and polydispersity index.

As noted above, the present invention contemplates downstream applications of the CLEAs of the present invention. For example, the CLEAs may be immobilized on a solid support, such as the membrane of a membrane microreactor, which can then be used in the production of a biochemical product via the catalytic activity of the immobilized enzyme(s).

Accordingly, the present invention further provides a method for the preparation of a solid support comprising at least one immobilized enzyme, the method comprises the steps of a) obtaining at least one (such as a plurality of) CLEA according to the present invention, and b) immobilizing the CLEA(s) on or within a solid support.

Non-limiting examples of a solid support include membranes, porous or non-porous beads, porous or non-porous particles, hollow fibers, monolithic columns, microchannels or microchambers. According to some embodiments, the solid support is a membrane, such as an ultrafiltration membrane. According to some embodiments, the solid support is a hollow fiber. According to some embodiments, the solid support is a monolithic column. According to some embodiments, the solid support is a microchannel. According to some embodiments, the solid support is a microchamber.

According to some embodiments, the solid support is one or more (such as a plurality of) porous or non-porous particle(s). The porous or non-porous particle(s) may have a diameter between about 50 nm and about 1 mm. The porous or non-porous particle(s) can be magnetic.

According to some embodiments, the solid support is one or more (such as a plurality of) porous or non-porous bead(s). Porous or non-porous beads can be applied to a stirred tank bioreactor, perfusion bioreactor, packed bed bioreactor, fluidized bed bioreactor, spinning disc bioreactor or the like.

The present invention provides a method for the preparation of a membrane microreactor, hollow fiber bioreactor, packed bed reactor, monolithic column, microchannel or microchamber comprising at least one immobilized enzyme, the method comprises the steps of a) obtaining at least one (such as a plurality of) CLEA according to the present invention, and b) immobilizing the CLEA(s) on or within a surface of a membrane microreactor, hollow fiber bioreactor, packed bed reactor, monolithic column, microchannel or microchamber.

According to some embodiments, the present invention provides a method for the preparation of a membrane microreactor comprising at least one immobilized enzyme, the method comprises the steps of a) obtaining at least one (such as a plurality of) CLEA according to the present invention, and b) immobilizing the CLEA(s) on or within the membrane surface of a membrane microreactor. Optionally, the method(s) above comprises c) washing the immobilized CLEA particles. The washing step is primary intend to remove residual amounts of the precipitating agent and cross-linking agent to prevent further crosslinking and deactivation of the prepared CLEA.

Membrane microreactor per se are well know to the skilled person, and have been described in the scientific literature, for example, in Handbook of Membrane Reactors, Volume 2—Reactor Types and Industrial Applications—Woodhead Publishing Series in Energy, 2013, especially in Chapter 5 titled “Microreactors and membrane microreactors: fabrication and applications”, or in Kiani, M. R., Meshksar, M., Makarem, M. A. et al. Catalytic Membrane Micro-Reactors for Fuel and Biofuel Processing: A Mini Review. Top Catal (2021), the contents of which are hereby incorporated by reference.

As described by Kiani et al., microreactors are a type of microfluidic device with a submillimeter range of dimensions (the range of length-width-diameter-height are usually 10-1000 μm). In general point of view, microreactors are designed in different structures and shapes from glass, silicon, or metallic substances for having proper mixing and reaction mechanisms. The advantages of micro-reactors and membrane separation which are combined in membrane micro-reactors (MMR) listed as improved heat and mass transfer, high surface area to volume ratio, enhanced catalytic efficiency with no equilibrium limitation, flexibility in reactor design, a short distance of molecular diffusion as well as high operational safety. By the use of membrane microreactors, the process efficiency will be enhanced as in these types of reactors, the chemical reaction and separation processes are integrated into one unit which causes a reduction in reaction steps. The membrane systems have been known due to their ability in controlling two-phase components mixing, considerable surface area per volume, and high selectivity.

Generally, the key component of a membrane microreactor is its membrane onto which the CLEAs of the present invention are immobilized and which separates the enzyme(s) and reactant(s)/produce(s) in different phases. Different membrane types may be used in the membrane microreactor including, but not limited to, microfiltration membranes, ultrafiltration membranes and nanofiltration membranes. Preferably, the membrane is an ultrafiltration membrane, for example an ultrafiltration membrane have a molecular weight cut off (MWCO) ranging from about 50 kDA to about 500 kDA, such as from about 100 kDA to about 300 kDA. Non-limiting examples of ultrafiltration membranes are the Biomax® membranes from Millipore Amicon® Bioseparations, Jaffrey, USA.

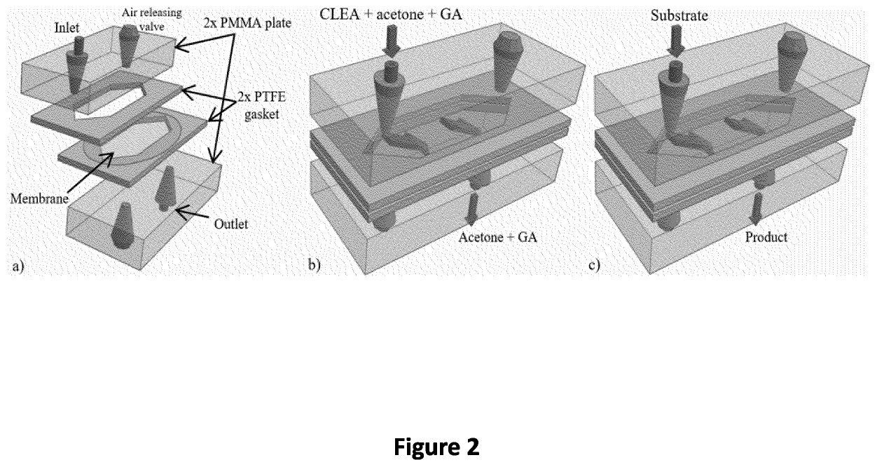

The membrane microreactor may, for example, comprises two poly(methylmethacrylate) (PMMA) plates with inserted polytetrafluoroethylene (PTFE) spacers and said membrane. A schematic, non-limiting presentation of such membrane microreactor is shown in FIG. 2.

The membrane microreactor may have one or more inlets and one or more outlets through which the CLEA particles as well as downstream reactants can be in introduced, respectively contaminants and products can exit the system.

By way of example, during the immobilization and purification of the CLEA particles, a CLEA suspension is introduced into the membrane microreactor through an inlet of the microreactor and the particles are anchored on the membrane surface while residual precipitating agent and crosslinking agent flow through the membrane and exited the microreactor at an outlet. The CLEA suspension may be added to the membrane microractor at a flow rate ranging from about 50 μL/min to about 300 μL/min, such as at a flow rate ranging from about 100 μL/min to about 200 L/min. After the initial anchoring of CLEA inside the membrane microreactor, a washing solution, such as a PBS solution, may be introduced, e.g., at a flow rate ranging from about 100 μl/min to about 200 μL/min, at the inlet to flush the remaining precipitating agent and crosslinking agent out of the device to prevent further crosslinking and deactivation of the prepared CLEA.

As a result of the method, the present invention thus provides a solid support on or within at least one (such as a plurality of) CLEA according to the present invention is (are) immobilized.

The present invention further provides a membrane microreactor, hollow fiber bioreactor, packed bed reactor, monolithic column, microchannel or microchamber characterized in that at least one (such as a plurality of) CLEA according to the present invention is (are) immobilized on or within the surface of the membrane microreactor, hollow fiber bioreactor, packed bed reactor, monolithic column, microchannel or microchamber.

According to some embodiments, the present invention provides a membrane microreactor characterized in that at least one (such as a plurality of) CLEA according to the present invention is (are) immobilized on or within the membrane surface of the membrane microreactor.

The present invention further contemplates an integrative microfluidic system combining the CLEA preparation, in situ immobilization and optionally cleaning, which allows an integrated process for biochemical synthesis.

The present invention thus provides a microfluidic system comprising a first reaction region, a second reaction region, which is in fluidic connection with the first reaction region, and an immobilization region, such as a membrane microreactor, which is in fluidic connection with the second reaction region.

The immobilization region may be a membrane microreactor, a hollow fiber bioreactor, a packed bed reactor, a monolithic column, a microchannel or a microchamber. The microchannel may be a microchannel chip, a microchannel plate, a microchannel tube or a capillary.

Preferably, the immobilization region is a membrane microreactor. The present invention thus provides a microfluidic system comprising a first reaction region, a second reaction region, which is in fluidic connection with the first reaction region, and a membrane microreactor, which is in fluidic connection with the second reaction region.

It is understood that the details about the microfluid system given above in the context of the methods of the present invention apply mutatis mutandis.

For example, the microfluidic system of the present invention may have multiple inlets through which reactants can be injected into the system. Suitable, each inlet may be in fluidic connection with at least one injection means, such as a microfluidic pressure pump (e.g., syringe pump, peristaltic pump or piezoelectric pump), either directly or indirectly via one or more micromixers, such as a T- or Y-micromixer(s). Preferably, the injection means is a syringe pump.

Thus, according to some embodiments, the first reaction region comprises at least one inlet which is in fluidic connection with at least one injection means, such as at least one microfluidic pressure pump, such as at least one syringe pump.

According to some embodiments, the at least one injection means is in fluidic connection with the at least one inlet of the first reaction region via a first micromixer.

According to some embodiments, the first micromixer is in fluidic connection with a first injection means and a second injection means, such as a first microfluidic pressure pump and a second microfluidic pressure pump, such as a first syringe pump and a second syringe pump.

According to some embodiments, the microfluidic system of the present invention is characterized in that at least one (such as a plurality of) cross-linked enzyme aggregate (CLEA) is (are) immobilized on or within the surface of a membrane microreactor, hollow fiber bioreactor, packed bed reactor, monolithic column, microchannel or microchamber.

According to some embodiments, the microfluidic system of the present invention is characterized in that at least one (such as a plurality of) CLEA according to the present invention is (are) immobilized on or within the surface of a membrane microreactor, hollow fiber bioreactor, packed bed reactor, monolithic column, microchannel or microchamber.

According to some embodiments, the microfluidic system of the present invention is characterized in that at least one (such as a plurality of) cross-linked enzyme aggregate (CLEA) is (are) immobilized on the membrane surface of the membrane microreactor.

According to some embodiments, the microfluidic system of the present invention is characterized in that at least one (such as a plurality of) CLEA according to the present invention is (are) immobilized on the membrane surface of the membrane microreactor.

The present invention further provides a process for producing a chiral amine comprising the step of catalyzing a transamination reaction using an amine transaminase cross-linked enzyme aggregate (ATA-CLEA) on a ketone compound and an amino donor, wherein the ATA-CLEA is a ATA-CLEA obtainable by the method according to the present invention.

According to some embodiments, the process is carried out on a membrane microreactor as detailed herein, wherein at least one (such as a plurality of) ATA-CLEA obtainable by the method according to the present invention is immobilized on the membrane surface of the membrane microreactor.

When a numerical value is preceded by the term “about”, the term “about” is intended to indicate+/−10%.

As used herein, the indefinite articles “a” and “an” mean “at least one” or “one or more” unless the context clearly dictates otherwise.

As used herein, the terms “comprising”, “including”, “having” and grammatical variants thereof are to be taken as specifying the stated features, steps or components but do not preclude the addition of one or more additional features, steps, components or groups thereof. Further, the use of “comprising” and “comprises”, is to be understood as also disclosing “consisting of” and “consists of” respectively.

Where a numerical limit or range is stated herein, the endpoints are included. Also, all values and sub ranges within a numerical limit or range are specifically included as if explicitly written out.

Having generally described this invention, a further understanding can be obtained by reference to certain specific examples, which are provided herein for purposes of illustration only, and are not intended to be limiting unless otherwise specified.

### EXAMPLES

The aim of our research was to develop a microfluidic platform for the controlled production of CLEAs, in which precipitation and crosslinking occur in a sequential step in flow. In this way, the concentration of precipitant and crosslinker as well as the flow rates could be optimized in terms of CLEA particle size, size distribution, and enzyme activity recovery. Furthermore, the in situ immobilization of synthesized CLEA particles in a membrane microreactor with two membranes of different pore size was investigated to develop an integrated process for amine transaminase cross-linked enzyme aggregates (ATA-CLEAs) synthesis and further application in a continuously operated transamination in a microfluidic device.

### 1. EXPERIMENTAL

### 1.1 Materials

Acetone, acetophenone (ACP), glutaraldehyde (GA), isopropanol, (S)-a-methylbenzyl amine (S-α-MBA), sodium pyruvate (PYR), pyridoxal-5′-phosphate (PLP) and sodium hydroxide were purchased from Sigma-Aldrich (St. Louis, USA). Acetonitrile was purchased from Lab Honeywell (Seelze, Germany), and lyophilized amine transaminase (ATA-v1) was donated by c-LEcta GmbH, Leipzig, Germany. Ultrafiltration membranes (Biomax®, molecular weight cut-off, MWCO, 100 and 300 kDa) were purchased from Millipore Amicon® Bioseparations, Jaffrey, USA.

### 1.2 Preparation of Amine-Transaminase CLEA Particles Using a Microfluidic System

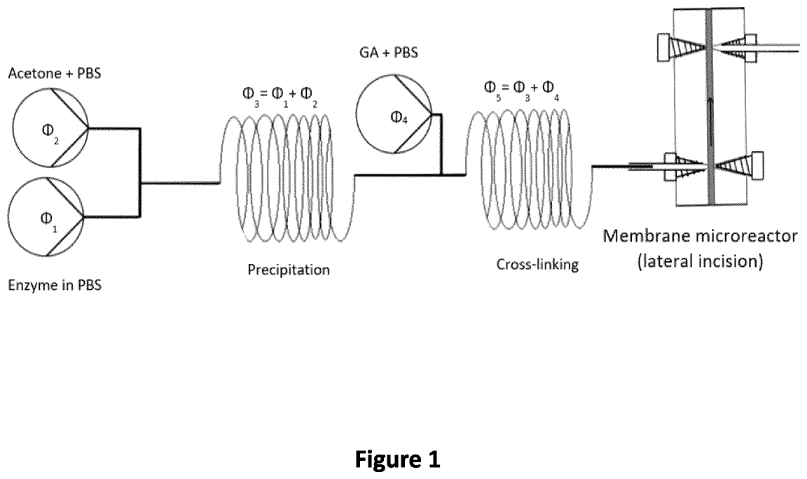

The microfluidic system used to prepare CLEA particles consisted of three syringe inlets, each connected with its own pump, two fluoroethylene propylene (FEP) microtubes (ID 794 μm, OD 1.58 mm, each tube 8.8 m long), two T-shaped polyether ether ketone (PEEK) micromixers, and a microscope with a high-speed camera (Motion Scope, Ljubljana, Slovenija). Phosphate buffer solutions (PBS, 20 mM, pH 8) of a lyophilized enzyme (4 mg mL⁻¹) and acetone (from 6.25 to 100 vol. %) were added separately to the T-micromixer using two syringe pumps at equal flow rates of 50 μL min⁻¹. All inlet solutions were filtered through 0.45 μm filters before use. As evident from FIG. 1, enzyme precipitation took place in the FEP tube before the PBS solution containing glutaraldehyde (GA) was added at concentrations ranging from 3.125 to 50 mM. The latter was introduced through another T-micromixer using syringe pump at a flow rate of 100 μl min⁻¹, and the crosslinking took place in a second FEP tube (FIG. 1).

The outlet of the second tube was collected in a collection vessel containing PBS, which was placed on a magnetic stirrer to prevent particle clogging of and further cross-linking.

The prepared CLEAs were stored in PBS and were further analyzed for particle size and recovered enzyme activity.

To compare this system with the previously reported microfluidic CLEA preparation, acetone and GA PBS solutions were introduced along with the enzyme solution, as indicated in the Supporting information.

### 1.2.1 Preparation of Amine-Transaminase CLEA Particles Using a Conventional Batch System (Reference).

The conventional batch preparation of CLEA particles consisted of mixing various concentrations of acetone with enzyme solution in 12 mL centrifuge tubes via magnetic mixer. Acetone (from 25 to 90 vol. %) and enzyme (1 mg mL-1) in phosphate buffer solution (PBS, 20 mM, pH 8) were added in 9:1 ratio. All inlet solution were filtered through 0.45 μm filters before use. The mixtures were mixing at 1000 min-1 at either 4° C. or 23° C. to promote enzyme precipitation. After 1 h, glutaraldehyde solution was added in a concentration ranging from 3.125 to 50 mM to initiate the crosslinking process. After additional 1 h, the CLEA's formed were centrifuged at 5000 g for 20 min and washed twice with the PBS buffer.

1.3 Preparation of the Membrane Microreactor with Immobilized Enzyme

As indicated in FIG. 1, a membrane microreactor was integrated into the system for ATA-CLEA production. The membrane microreactor presented in FIG. 2a consists of two poly(methyl methacrylate) (PMMA) plates with inserted 200 μm thick polytetrafluoroethylene (PTFE) spacers and an ultrafiltration membrane with 100 kDa or 300 kDa MWCO. The membrane was adhered to the PTFE spacer with double-sided adhesive tape. Each PMMA plate had two holes to inserting the PEEK connectors. The depth of the microreactor, defined by the top PTFE gasket, was 200 μm and the available surface area of the membrane was 700 mm², corresponding to a microreactor volume of 140 μL. One of the holes on the top of the device was used to introduce CLEA particles in the PBS mixture with acetone and GA or substrates for biotransformation, and the other was used to remove the air bubbles from the device (FIG. 2a). During the immobilization and purification of the CLEA particles, the CLEA suspension was introduced through the FEP tube at the top of the reactor and the particles were anchored on the membrane surface while acetone and GA flowed through the membrane and exited the microreactor at the bottom exit (FIG. 2b). Flow rate through the microreactor was in all cases 200 μL min⁻¹, while the pumping time was different and was 1.3 min for 100 kDa membrane, and 9.28 min for the membrane with 300 kDa MWCO. After the initial anchoring of CLEA inside the membrane microreactor, approximately 5 mL of PBS solution was introduced at a flow rate of 200 μl min⁻¹ at the inlet port to flush the remaining acetone and GA out of the device to prevent further crosslinking and deactivation of the prepared CLEA catalyst.

The enzyme concentration in the membrane microreactor, C_{μr} [mg mL⁻¹] was calculated using Equation 1:

$${C}_{μ r} = \frac{t \cdot {Φ}_{5} \cdot ( {C}_{e} - ( \frac{{a}_{out} \cdot {V}_{out}}{{a}_{spec} \cdot {V}_{μ r}} ) )}{{V}_{μ r}} \qquad ( 1 )$$

where φ₅ is the total volumetric flow rate in the second tube, t [min] is the pumping time of the solution through the membrane microreactor, Cₑ [mg mL⁻¹] is the enzyme concentration in the second tube (typically 1 mg mL⁻¹), aₒᵤₜ is the enzyme activity in the outlet solution [U mL⁻¹], a_{spec} is the specific enzyme activity [U mg⁻¹], Vₒᵤₜ [mL] is the volume of the outlet solution, and V_{μr} [mL] is the volume of the microreactor above the membrane.

To evaluate the activity of ATA-v1 immobilized on a membrane microreactor without prior CLEA formation (nonaggregated ATA-v1), no precipitant or crosslinker was added to the stream. The eluat solution was subjected to a batch activity assay. Immobilization yield was calculated based on the ratio of the immobilized enzyme activity in the membrane reactor, calculated from the enzyme activity introduced to the reactor (offered activity) and subtracting the activity at the microreactor outlet, and offered enzyme activity.

### 1.4 Biotransformation in a Membrane Microreactor

Biotransformation was performed in a 100-kDa and 300-kDa membrane microreactor as shown in FIG. 2c. The 40 mM PBS equimolar solution of both substrates containing 0.1 mM PLP was introduced at flow rates between 50 and 200 μl min-1 into the membrane microreactor through the inlet, while the outlet solution was collected at the bottom and analyzed as described below. The entire reactor was immersed in a water bath at 50° C. to ensure isothermal operating conditions.

Gross yield GY [%], space-time yield STY [U mL⁻¹ or mmol L⁻¹ h⁻¹], biocatalyst productivity P_{b-μr} [U mg⁻¹] and immobilization efficiency η [%] were determined using Equations 2, 3, 4 and 5:

$$G Y = \frac{{C}_{ACP}}{{C}_{MBA - in}} \cdot 100 \qquad ( 2 )$$

$$S T Y = \frac{{C}_{ACP}}{τ} \qquad ( 3 )$$

$${P}_{b - μ r} = \frac{{C}_{ACP}}{τ \cdot {C}_{μ r}} \qquad ( 4 )$$

$$η = \frac{S T Y}{{C}_{μ r} \cdot {a}_{spec}} \cdot 100 \qquad ( 5 )$$

where τ [min] is residence time in a membrane microreactor, C_{ACP} [mmol mL⁻¹] is ACP concentration at the microreactor outlet, while C_{MBA-in} [mmol mL⁻¹] is MBA inlet concentration.

Operational stability of a 100-kDa membrane microreactor was tested by continuously pumping the microreactor with 40 mM PBS equimolar solution of both substrates containing 0.1 mM PLP at the flow rate of 5 μL min⁻¹ for 6 days.

### 1.5 Analytical Methods

### 1.5.1 Particle Size Measurement

Particle size was measured in the colloidal suspension collected at the exit of the second tube of the microfluidic system used to produce CLEA particles. The Litesizer™ 500 particle size analyzer (Anton-Paar, Graz, Austria) with dynamic light scattering (DLS) was applied. The wavelength of the laser was 658 nm and power of 40 mW. A standard operating procedure was established for the measurement of CLEA particles in water at 20° C. Each sample was measured 10 times for 30 s. The average particle radius was calculated as an average value of the 10 measurements and is reported with standard deviation and polydispersity index.

To determine particle size for CLEA prepared in batch mode, because of the size (micro scale), a confocal laser scanning microscope (Zeiss LSM 700, Oberkochen, Germany) was used. Approximately 150 particles for each sample were examined through image analysis to determine the mean particle size and the PDI (polydispersity index) as well as Feret ratio which gives an indication for the elongation of the particle.

### 1.5.2 Determination of Free Enzyme and CLEA Activities in Batch Experiments

The activity of the free enzyme and CLEAs was determined in a batch experiment in a water bath at 30° C. 3 mL of 20 mM PBS (pH 8) were mixed with 1 ml of 200 mM MBA, 400 μL 500 mM PYR, and 100 μL of 0.1 mM PLP, yielding final concentrations of 40 mM for MBA and PYR, and 0.1 mM for PLP cofactor. 0.5 mL of the free enzyme solution or CLEA suspension was added to obtain a final enzyme concentration of 0.0235 mg mL-1. All solutions were thermostated to 30° C. before biotransformation. The activity of free ATA-v1 and ATA-CLEA was also tested at different temperatures between 30° C. and 70° C. 200 μl samples were taken at 0, 2.5, 5, 10, 20, 40, and 60 min, and mixed with 800 μL of 0.1 M sodium hydroxide to quench the reaction and analyzed as specified below. Enzyme activity [U] was estimated from the linear part of the curve plotting product concentration vs process time, and reagent solution volume. 1 U was defined as 1 μmol of product (ACP) formed per min. The specific enzyme activity a_{spec} [U mg⁻¹] was calculated based on the enzyme mass used in the assay. Recovered activity [%] was determined using Equation 6:

$$Recovered activity = \frac{{a}_{CLEA}}{{a}_{free}} \cdot 100 \qquad ( 6 )$$

where a_{CLEA} [U] is the activity of ATA-CLEA and a_{free} [U] is the activity of free (nonaggregated) enzyme ATA-v1.

### 1.5.3 HPLC Analysis

Samples from batch and microreactor-based biotransformation were analyzed using HPLC instrument (Shimadzu, Tokyo, Japan) with a Gemini 5μ C18 column of 150×4.6 mm (Phenomenex, Torrance, USA) and a UV-VIS detector, with MBA and ACP detected at a wavelength of 226 nm. The mobile phase was a mixture of MiliQ water with 1 mM NaOH (pH 11) and acetonitrile in a volume ratio of 1:1. The volumetric flow rate was 1 ml min⁻¹, the measurement time was 5 min, and the oven temperature was 30° C. The retention time of MBA and ACP was 2.61 min and 3.77 min, respectively.

### 2. RESULTS AND DISCUSSION

### 2.1 Preparation and Characterization of ATA-CLEA Particles

A novel microfluidic system for the preparation of CLEA particles has been developed that allows separate precipitation and crosslinking of the agglomerates. This differs from previously reported microfluidic CLEA preparation systems where the precipitation and crosslinking solution were introduced through the same inlet (Nguyen & Yang, 2014; Jannat & Yang, 2020). This configuration was also tested and compared with the results of the newly developed setup. As shown in Figure S1, sequential crosslinking with 3 inlets resulted in a more homogeneous ATA-CLEA particle size distribution compared to simultaneous precipitation and crosslinking using 2 inlets and the same precipitant and crosslinker concentrations as in the system with 3 inlets. Acetone was used for ATA-v1 precipitation in the flow, as it resulted in the best enzyme immobilization yield and recovered activity among several precipitants applied in a batch ATA-CLEA preparation (Velasco-Lozano et al., 2020), while GA was used as a widely adopted crosslinker. Measurement of the particle size distribution revealed that the sequential steps system resulted in an absence of macromolecules below 10 nm radius, which would be below the range of a homotetrameric ATA-v1 active enzyme conformation (Borner et al., 2017). Therefore, we could conclude that all enzymes that entered the microflow system were crosslinked in ATA-CLEAs, which was considered when evaluating CLEA enzyme concentration using Equation 1.

In preliminary experiments (data not shown), flow rates ensuring efficient enzyme precipitation in the first tube and crosslinking in the second tube were set to be 100 μl min⁻¹ and 200 μL min⁻¹, respectively. Further studies focused on optimizing the conditions for the preparation of the ATA-CLEA, i.e., the concentration of enzyme, acetone and GA. At 12.5 mM GA and 50% acetone concentration, 2.5 mg mL⁻¹ enzyme in the second tube (Cₑ) resulted in an average particle radius of 243.59±27.77 with a polydispersity index of 0.27.

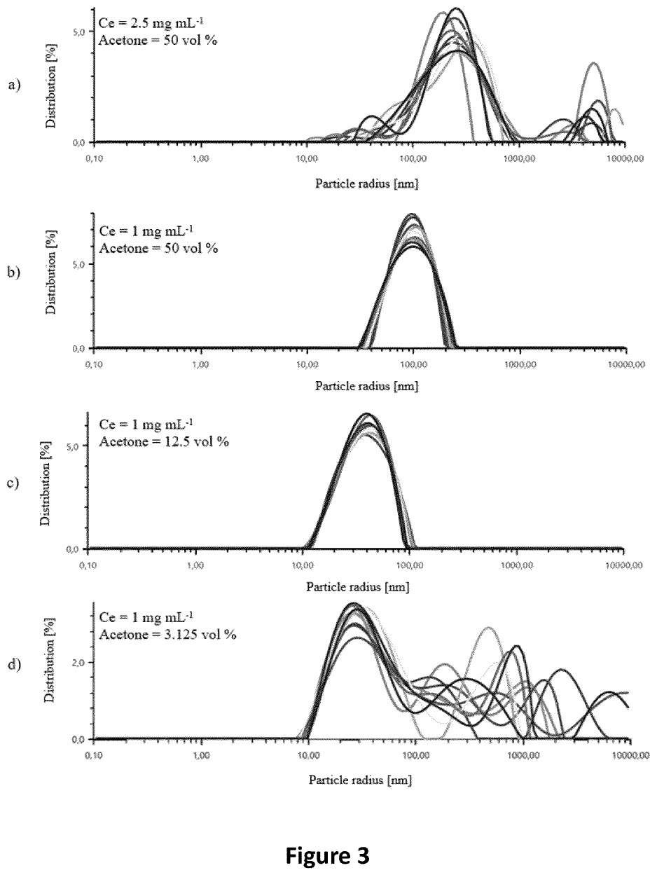

The heterogenous particle size distribution with two main peaks, the second of which is in the range of several μm, can be seen in FIG. 3a, which shows DLS analysis of the sample. Using enzyme concentrations above 2.5 mg mL⁻¹ under the same conditions resulted in the formation of very large particles that clogged the tube, while lowering the enzyme concentration to 1 mg mL⁻¹ resulted in much smaller and more uniform particles with an average particle radius of 93.06±0.94 nm and a polydispersity index of 0.2 (FIG. 3b). Therefore, this enzyme concentration was used in further studies.

As evident from FIG. 3c, a decrease in acetone concentration from 50 to 12.5 vol. % resulted in the formation of uniform and significantly smaller particles with an average radius of 37.13±0.38 nm and a polydispersity index of 0.19. A similar decrease in particle size with a decrease in precipitant (acetonitrile) concentration was reported in the studies performed by Nguyen & Yang, 2014, and by Jannat & Yang, 2020, in which the size of the synthesized catalase-CLEA particles decreased to about half when precipitant concentration was reduced from 100 to 25 vol. %. However, in our study, further decreasing the acetone concentration to 3.125 vol. % resulted in significantly less homogeneity of the generated ATA-CLEAs, as shown in FIG. 3d, and larger particles with an average radius of 172.68±101.74 and a polydispersity index of 0.27.

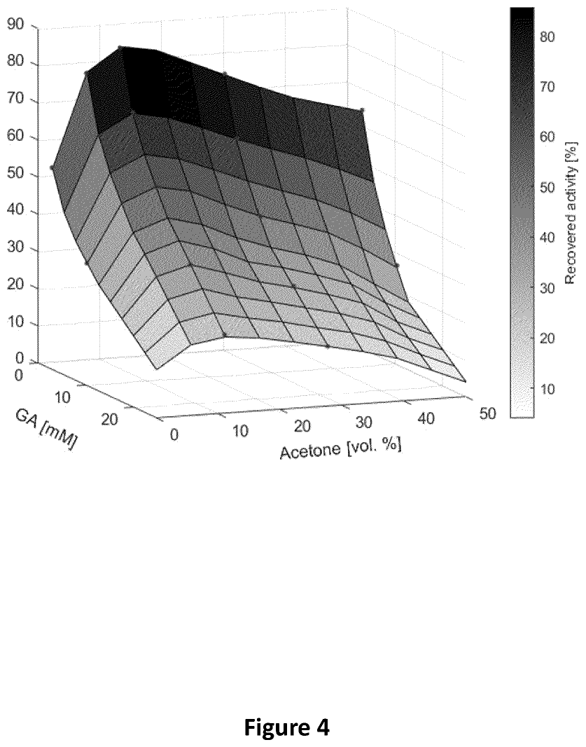

In addition to the particles size, the evaluation of the recovered enzyme activity is crucial to assess the applicability of the formed CLEAs. The recovered activities of the ATA-CLEA particles were calculated from the activities of the free enzyme and the ATA-CLEAs based on Equation 6. A comparison of the recovered activities of ATA-CLEAs prepared at 1 mg mL⁻¹ enzyme and at various acetone and GA concentrations is presented in FIG. 4. There, the experimentally determined recovered activities of ATA-CLEAs prepared in a microfluidic system at various GA and acetone concentrations are presented in a 3D plot with interpolation between the experimentally determined values. From FIG. 4, in can be seen that the concentration of crosslinking agent has a greater effect on the recovered enzyme activity than the precipitant above 12.5 vol. % of acetone. The highest ATA-CLEA recovered activities were obtained at the lowest tested GA concentration of 3.125 mM. Increasing the GA concentration to 25 mM at the same acetone concentration resulted in a 66.2% decrease in recovered activity. The decrease in biocatalyst's recovered activity at higher crosslinker concentration could be attributed to the increased inaccessibility of the enzyme's active site to the substrate and the hindered enzyme's flexibility due to the higher degree of crosslinking. An optimum of 87.1% retained activity was observed at acetone concentration of 12.5 vol. % and GA concentration of 3.125 mM. This is much higher than reported for ATA-CLEAs prepared in a batch synthesis, where the highest recovered activities of 36% was obtained at 50 mM GA, 90 vol. % acetone, and with a co-immobilization of 100 mg mL-1 bovine serum albumin (Velasco-Lozano et al., 2020).

Decreasing the acetone concentration from 50 to 12.5 vol. % at the same GA concentration of 3.155 mM resulted in an increase in recovered activity of 23.3% (FIG. 4). This is consistent with the smaller size and higher homogeneity of ATA-CLEA particles obtained at 12.5 vol. % acetone concentration, as shown in FIG. 3c and discussed above. A similar trend was reported by Jannat & Yang, 2020, where a decrease in precipitant (acetonitrile) concentration improved the recovered activity of catalase-CLEA from 12 to 60%. However, in our study, further decrease of acetone concentration to 3.125 vol. % resulted in up to 37.1% lower recovered activities (FIG. 4), accompanied by non-homogeneity of the ATA-CLEAs formed (FIG. 3d). This may be attributed to poor enzyme precipitation at low acetone concentration and excessive crosslinking of the enzyme.

On the other hand, particle size was not affected by the concentration of GA (data not shown), suggesting that the crosslinking step does not affect the size of aggregates formed in the first tube. This agrees with the reports on catalase-CLEAs, where an increase in GA concentration resulted in a decrease in recovered activity, while the average particle size remained unchanged and was 153 nm (Jannat & Yang, 2020).

For CLEA's prepared in batch mode (reference), particle size (as well as PDI) and retained activity was observed for particles prepared with different acetone and GA concentrations. The reason for batch CLEA preparation was to compare the results with microfluidic approach.

First observation is that, the particles are not formed when using acetone concentrations below 50 vol. %, and performing the precipitation step at room temperature. Furthermore, the acetone concentration did not seem to have an effect on retained activity. The particle size measurements were performed only on 90 vol. % acetone as a precipitant. It is clear that the resulted CLEA particles were much bigger than the ones prepared through microfluidics-32 μm (1000× bigger) with also much higher PDI of 0.43 (2.5× higher).

The highest retained activity achieved with batch prepared CLEA's is 36.7% for CLEA with 90 vol. % acetone and 3,125 mM GA. With increasing the GA concentration to 50 mM, the retained activity drops to 5.4%. The precipitant concentrations doesn't have an effect on retained activity.

### 2.2 Biotransformation in a Membrane Microreactor

Continuous transamination was performed in a microreactor with two microchambers separated by a 100 kDa or 300 kDa MWCO ultrafiltration membrane, as shown in FIG. 2. A nonaggregated ATA-v1 and ATA-CLEAs were immobilized on the membrane as described in Section 1.3.

When the 300-kDa membrane was tested for ATA-CLEAs immobilization efficiency, a pump time of 9.28 min was used, corresponding to Vₒᵤₜ of 1.86 mL. The activity measured in the outlet aₒᵤₜ was 0.0171 U mL⁻¹, which yields C_{μr} of 17.68 mg mL⁻¹. Considering the ATA-CLEA a_{spec} at 50° C. and V_{μr}, 34.65 U mL⁻¹ of immobilized enzyme activity in the membrane microreactor was calculated, corresponding to the immobilization yield of 99%. After performing the selected biotransformation in this microreactor at 50° C. and a flow rate of 50 μL min⁻¹ and analyzing the outlet MBA and ACP concentration, a STY of 330 mmol_{ACP} L⁻¹ h⁻¹ was calculated from Equation 3, corresponding to 5.5 U mL⁻¹. The immobilization efficiency calculated from Equation 5 was 15.87%.

To improve these results, a 100-kDa membrane was used and the enzyme load was reduced by decreasing the pump time to 1.3 min. From the measurement of enzyme activity in the eluate, which was below the detection limit, we can conclude that ATA-CLEA enzyme preparation was retained on this ultrafiltration membrane with an immobilization yield of 100%. The enzyme concentration in the 100-kDa membrane microreactor calculated according to Equation 1 was 1.86 mg mL⁻¹, corresponding to 3.64 U mL⁻¹ of ATA-CLEA activity. When biotransformation of MBA was performed at 50° C. and a flow rate of 50 μl min-1, the 100-kDa microreactor with the immobilized ATA-CLEA gave a STY of 149.31 mmol_{ACP} L⁻¹ h⁻¹ (2.49 U mL⁻¹), corresponding to a n of 60.5%, while biocatalyst productivity P_{b-μr} of 9.7 mg_{ACP} mg_{ATA}⁻¹ h⁻¹ was calculated using Equation 4. This is higher than reported for ATA immobilized on polymer-coated glass beads with controlled porosity (Ezig™) via iron affinity binding and used in a packed bed reactor, where the biocatalyst productivity was 2.99 mg_{ACP} mg_{ATA}⁻¹ h⁻¹ (Böhmer et al., 2019). Surface immobilization of ATA-wt, genetically fused to silica-binding Z_{basic2} protein and attached in a silicon/glass microchannel with an enzyme load of 1.57 U mL⁻¹ yielded P_{b-μr} of 16.7 mg_{ACP} mg_{ATA}⁻¹ h⁻¹ (Miložič et al., 2018). Bajić et al., 2017 reported on biocatalyst productivities between 0.17 and 16 mg_{ACP} mg_{ATA}⁻¹ h⁻¹ when performing the same reaction with a wild type ATA (ATA-wt) in a miniaturized packed bed reactor using enzyme loads between 40.57 and 167.45 U mL-1. On the other hand, when His-tagged ATA was immobilized on the surface of beads in a packed bed reactor, a biocatalyst productivity of 244 mg_{ACP} mg_{ATA}⁻¹ h⁻¹ was reported, but with only 8-11% recovered activity (Benítez-Mateos et al., 2018).

Immobilization of ATA-v1 on a 100-kDa membrane microreactor without prior CLEA formation (nonaggregated ATA-v1) was also performed with the same Cₑ, pump time, and flow rate. No enzyme activity was again detected in the eluate, so the enzyme load was again 1.86 mg mL⁻¹, corresponding to 4.11 U mL⁻¹. Biotransformation with nonaggregated ATA-v1, performed under the same conditions as described above, resulted in a STY of 88 mmol_{ACP} L⁻¹ h⁻¹ (1.47 U mL⁻¹), corresponding to an of 40.4%. Obviously, the nonaggregated ATA-v1 had a much lower immobilization efficiency than the ATA-CLEA preparation.

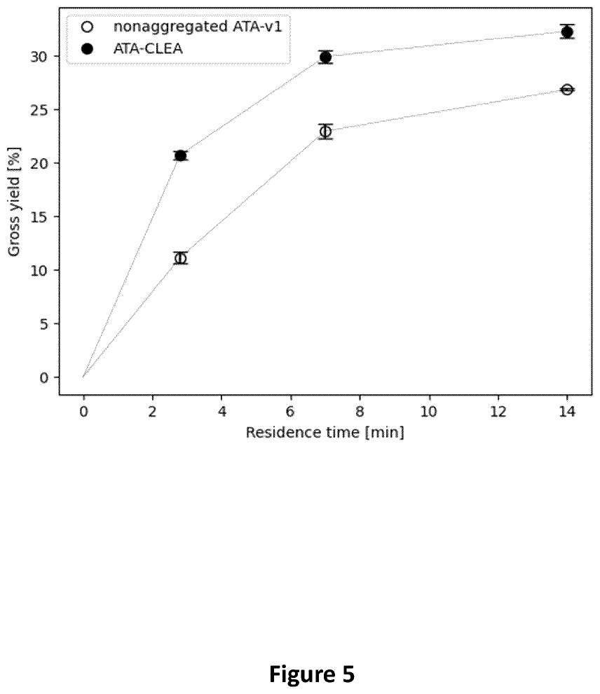

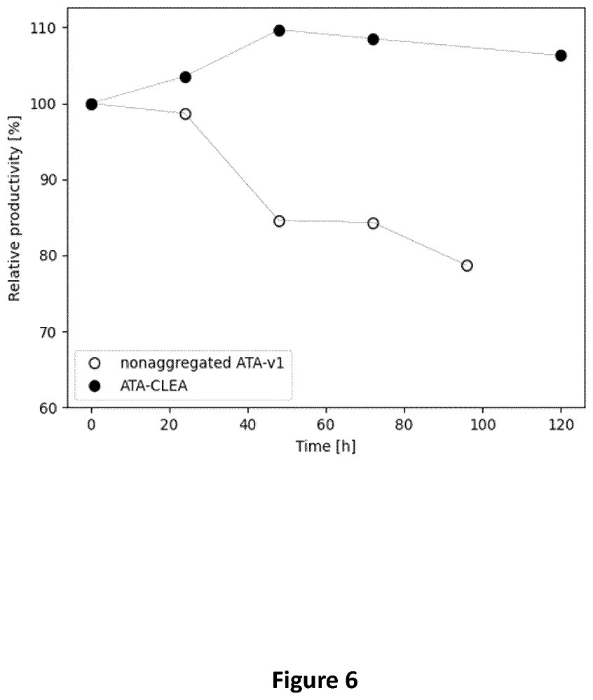

To determine the dependence of reaction yield on residence time in the membrane microreactor, an equimolar substrate solution containing PLP was pumped through the microreactor at 50° C. at various flow rates. The gross yield of ACP obtained with nonaggregated ATA-v1 and ATA-CLEA, immobilized in a 100-kDa membrane microreactor, was calculated based on Equation 2 and is shown in FIG. 6. As expected, longer residence times resulted in higher gross yields, reaching 32% at the residence time of 14 min with ATA-CLEA immobilized in a 100-kDa membrane. As evident from FIG. 5, ATA-CLEA immobilization in a membrane microreactor resulted in higher gross yields compared to nonaggregated ATA-v1 at all residence times tested. Using a 300-kDa membrane microreactor with higher loading of ATA-CLEA, as described above, a gross yield of 70% was achieved at a residence time of 21 min.

2.3 Operational Stability of a Membrane Microreactor with ATA-CLEA and Nonaggregated Enzyme

To evaluate the operational stability, a membrane microreactor containing enzymes immobilized on a 100 kDa MWCO membrane was continuously operated for 6 days at 50° C. and at a flow rate of 5 μl min-1, resulting in a residence time of 56 min. The nonaggregated ATA-v1 and ATA-CLEAs prepared with 12.5 vol % acetone and 3.125 mM GA containing the same amount of enzyme were tested. As evident from FIG. 6, ATA-CLEA particles exhibited higher stability than nonaggregated ATA-v1, as the productivity of ATA-CLEA was preserved for 6 days, while the productivity of the nonaggregated enzyme fell below 80% during the same period. This confirms previous results showing that CLEA aggregates are more robust and can maintain catalytic activity over a longer period of time (Sheldon & Woodley, 2018), mainly due to the rigidity of the crosslinked aggregates and their resistance to mechanical shear stress deformation in a continuous flow environment. Previous studies on ATA, entrapped or cross-linked in a porous polymer structures (Bajić et al., 2017 and Menegatti & Žnidaršič-Plazl, 2021) have also shown that relative productivity remained almost constant after more than 10 days. However, in the case of surface immobilization (Miložič et al., 2018) or immobilization on His-select nickel affinity agarose beads (Abdul Halim et al., 2013), relative productivity in flow-through reactors decreased to 10% after one day of continuous operation or to 60% after only 8 h of operation, respectively.

### 3. CONCLUSIONS

A novel approach for the synthesis of CLEA particles is presented in which precipitation and cross-linking occur in separate but interconnected parts of the microfluidic system. This method was successfully used for the selected ATA and resulted in CLEA nanoparticles of below 40 nm radius with very uniform particle size distribution and a high recovered activity of 87%. The method also reduced GA and acetone consumption and eliminated the need for an additional protein feeder usually used to capture the excess of GA. By optimizing acetone and GA concentration, a residual activity of 87.1% was obtained for ATA-CLEA, the highest reported so far for this enzyme.

The subsequent integration of a membrane microreactor into the microfluidic system enabled a one-step removal of residual precipitant and crosslinker as well as enzyme immobilization for further biotransformation. The use of 100 kDa and 300 kDa membranes resulted in immobilization yields of over 99% for both membranes. An enzyme loading of 3.64 U mL⁻¹ in the 100-kDa membrane microreactor yielded ATA-CLEA immobilization efficiency of 60.5% and fully maintained productivity within 6 days of continuous operation. This was much better than in the same microreactor with a nonaggregated ATA-v1, where an immobilization efficiency of 40.4% was obtained and a decrease in microreactor productivity to below 80% was observed within 6 days of continuous operation.

### LIST OF CERTAIN REFERENCES CITED IN THE DESCRIPTION

Abdul Halim, A., Szita, N., & Baganz, F. (2013). Characterization and multi-step transketolase-ω-transaminase bioconversions in an immobilized enzyme microreactor (IEMR) with packed tube. Journal of Biotechnology, 168(4), 567-575. https://doi.org/10.1016/j.jbiotec.2013.09.001
Aguilar, A., Twardowski, T., & Wohlgemuth, R. (2019). Bioeconomy for Sustainable Development. Biotechnology Journal, 14(8), 1-11. https://doi.org/10.1002/biot.201800638
Andrade, L. H., Kroutil, W., & Jamison, T. F. (2014). Continuous flow synthesis of chiral amines in organic solvents: Immobilization of E. coli cells containing both ω-transaminase and PLP. Organic Letters, 16(23), 6092-6095. https://doi.org/10.1021/ol502712v
Bajić, M., Plazl, I., Stloukal, R., & Žnidaršič-Plazl, P. (2017). Development of a miniaturized packed bed reactor with ω-transaminase immobilized in LentiKats®. Process Biochemistry, 52, 63-72. https://doi.org/10.1016/j.procbio.2016.09.021
Benítez-Mateos, A. I., Contente, M. L., Velasco-Lozano, S., Paradisi, F., & López-Gallego, F. (2018). Self-Sufficient Flow-Biocatalysis by Coimmobilization of Pyridoxal 5′-Phosphate and ω-Transaminases onto Porous Carriers. ACS Sustainable Chemistry and Engineering, 6 (10), 13151-13159. https://doi.org/10.1021/acssuschemeng.8b02672
Böhmer, W., Knaus, T., Volkov, A., Slot, T. K., Shiju, N. R., Engelmark, K., & Mutti, F. G. (2019). Highly efficient production of chiral amines in batch and continuous flow by immobilized ω-transaminases on controlled porosity glass metal-ion affinity carrier. Journal of Biotechnology, 291, 52-60. https://doi.org/10.1016/j.jbiotec.2018.12.001
Börner, T., Rämisch, S., Bartsch, S., Vogel, A., Adlercreutz, P., & Grey, C. (2017). Three in One: Temperature, Solvent and Catalytic Stability by Engineering the Cofactor-Binding Element of Amine Transaminase. Chembiochem, 18 (15), 1482-1486. https://doi.org/10.1002/cbic.201700236
Di Cosimo, R., Mc Auliffe, J., Poulose, A. J., & Bohlmann, G. (2013). Industrial use of immobilized enzymes. Chemical Society Reviews, 42 (15), 6437-6474. https://doi.org/10.1039/c3cs35506c
Guisan, J. M., López-Gallego, F., Bolivar, J. M., Rocha-Martin, J., & Fernandez-Lorente, G. (2020). The Science of Enzyme Immobilization. Methods in Molecular Biology, 2100(February), 1-26. https://doi.org/10.1007/978-1-0716-0215-7_1
Jannat, M., & Yang, K. L. (2020). A Millifluidic Device with Embedded Cross-Linked Enzyme Aggregates for Degradation of H2O2. ACS Applied Materials and Interfaces, 12(5), 6768-6775. https://doi.org/10.1021/acsami.9b21480
Kelly, S. A., Pohle, S., Wharry, S., Mix, S., Allen, C. C. R., Moody, T. S., & Gilmore, B. F. (2018). Application of ω-Transaminases in the Pharmaceutical Industry. Chemical Reviews, 118, 349-367. https://doi.org/10.1021/acs.chemrev.7b00437
Kiani, M. R., Meshksar, M., Makarem, M. A. et al. Catalytic Membrane Micro-Reactors for Fuel and Biofuel Processing: A Mini Review. Top Catal (2021). https://doi.org/10.1007/s11244-021-01505-1Malik, M. S., Park, E. S., & Shin, J. S. (2012). Features and technical applications of ω-transaminases. Applied Microbiology and Biotechnology, 94(5), 1163-1171. https://doi.org/10.1007/s00253-012-4103-3
Menegatti, T., & Žnidaršič-Plazl, P. (2021). Hydrogel-Based Enzyme and Cofactor Co-Immobilization for Efficient Continuous Transamination in a Microbioreactor. Frontiers in Bioengineering and Biotechnology, 9 (November), 1-10. https://doi.org/10.3389/fbioe.2021.752064
Miložič, N., Stojkovič, G., Vogel, A., Bouwes, D., & Žnidaršič-Plazl, P. (2018). Development of microreactors with surface-immobilized biocatalysts for continuous transamination. New Biotechnology, 47 (May), 18-24. https://doi.org/10.1016/j.nbt.2018.05.004
Nguyen, L. T., Seow, N., & Yang, K. L. (2017). Hollow cross-linked enzyme aggregates (h-CLEA) of laccase with high uniformity and activity. Colloids and Surfaces B: Biointerfaces, 151, 88-94. https://doi.org/10.1016/j.colsurfb.2016.12.006
Nguyen, L. T., & Yang, K. L. (2014). Uniform cross-linked cellulase aggregates prepared in millifluidic reactors. Journal of Colloid and Interface Science, 428, 146-151. https://doi.org/10.1016/j.jcis.2014.04.033
Päiviö, M., & Kanerva, L. T. (2013). Reusable ω-transaminase sol-gel catalyst for the preparation of amine enantiomers. Process Biochemistry, 48 (10), 1488-1494. https://doi.org/10.1016/j.procbio.2013.07.021
Savile, C. K., Janey, J. M., Mundorff, E. C., Moore, J. C., Tam, S., Jarvis, W. R., Colbeck, J. C., Krebber, A., Fleitz, F. J., Brands, J., Devine, P. N., Huisman, G. W., & Hughes, G. J. (2010). Biocatalytic asymmetric synthesis of chiral amines from ketones applied to sitagliptin manufacture. Science, 329 (5989), 305-309. https://doi.org/10.1126/science.1188934
Sheldon, R. A. (2019). Cleas, combi-cleas and ‘smart’ magnetic cleas: Biocatalysis in a bio-based economy. Catalysts, 9 (3), 1-31. https://doi.org/10.3390/catal9030261
Sheldon, R. A., Basso, A., & Brady, D. (2021). New frontiers in enzyme immobilisation: robust biocatalysts for a circular bio-based economy. Chemical Society Reviews. https://doi.org/10.1039/d1cs00015b
Sheldon, R. A., & Woodley, J. M. (2018). Role of Biocatalysis in Sustainable Chemistry. Chemical Reviews, 118, 801-838.
Van Den Biggelaar, L., Soumillion, P., & Debecker, D. P. (2017). Enantioselective transamination in continuous flow mode with transaminase immobilized in a macrocellular silica monolith. Catalysts, 7 (2). https://doi.org/10.3390/catal7020054
Velasco-Lozano, S., Jackson, E., Ripoll, M., López-Gallego, F., & Betancor, L. (2020). Stabilization of ω-transaminase from Pseudomonas fluorescens by immobilization techniques. International Journal of Biological Macromolecules, 164, 4318-4328. https://doi.org/https://doi.org/10.1016/j.ijbiomac.2020.09.003
Žnidaršič-Plazl, P. (2019). The Promises and the Challenges of Biotransformations in Microflow. In Biotechnology Journal (Vol. 14, Issue 8). https://doi.org/10.1002/biot.201800580
Žnidaršič-Plazl, P. (2021). Biocatalytic process intensification via efficient biocatalyst immobilization, miniaturization, and process integration. Current Opinion in Green and Sustainable Chemistry, 32, 100546. https://doi.org/10.1016/j.cogsc.2021.100546

1.-32. (canceled)

33. A method for the preparation of cross-linked enzyme aggregates (CLEAs), the method comprising the steps:
a) precipitation of at least one enzyme, optionally together with its required co-factor(s), in the presence of a precipitating agent; and
b) reacting the precipitated enzyme(s) with a crosslinking agent to obtain said CLEAs;
wherein said method is carried out in a microfluidic system, and
wherein steps a) and b) are not carried out simultaneously.

34. The method according to claim 33, wherein step a) comprises providing a buffered solution comprising a suitable amount of the enzyme and adding the precipitating agent.

35. The method according to claim 33, wherein the precipitating agent is selected from the group consisiting of ethanol, methanol, propanol, such as 1-propanol or 2-propanol, butanol, such as tert-butanol, acetonitrile, acetone, ethyl lactate, dimethoxyethane (DME), dimethylformamide (DMF), dimethyl sulfoxide (DMSO) and polyethylene glycol (PEG).

36. The method according to claim 33, wherein the precipitating agent is acetone.

37. The method according to claim 33, wherein the crosslinking agent is selected from the group consisting of glutaraldehyde, N-methylenebisacrylamide, bismaleimide, dextran, p-benzoquinone, glucoamylase and chitosan.

38. The method according to claim 33, wherein the crosslinking agent is glutaraldehyde.

39. The method according to claim 33, wherein the microfluidic system comprises a first reaction region and a second reaction region, both reaction regions being in fluidic connection with each other.

40. The method according to claim 39, wherein step a) is carried out in the first reaction region and step b) is carried out in the second reaction region.

41. The method according to claim 39, wherein the solution comprising the at least one enzyme and the precipitating agent are added simultaneously to the first reaction region.

42. The method according to claim 41, wherein the solution comprising the at least one enzyme and the precipitating agent are added at equal flow rates ranging from about 25 L/min to about 100 μL/min.

43. The method according to claim 39, comprising transferring the precipitated enzyme(s) obtained in step a) from the first reaction region to the second reaction region, and adding the buffered solution comprising the crosslinking agent to the second reaction region to initiate step b).

44. The method according to claim 43, wherein the crosslinking agent is added to the second reaction region at a flow rate ranging from about 50 μL/min to about 200 μL/min, such as at a flow rate of about 100 μL/min.

45. The method according to claim 33, which is carried out in a microfluidic system.

46. The method according to claim 33, wherein the at least one enzyme is selected from the group consisting of transferases such as amine transaminases, oxidoreductases, such as oxidases, peroxidases, laccases, ketoreductases, imine reductases, and carbamoylases, dehydrogenases, hydrolases such as esterases, lactamases such as beta-lactameses, proteases, cellulases, lipases, aminopeptidases, nitrilases, xylanases, glycosylases, amidases, lyases, such as hydroxynitrile lyases, and aldolases.

47. The method according to claim 33, wherein the enzyme is a transaminase.

48. The method according to claim 47, wherein the enzyme is an amine transaminase.

49. Cross-linked enzyme aggregate (CLEA) obtainable by the method according to claim 33, having a particle size distribution (d50) ranging from about 30 nm to about 100 nm.

50. The CLEA according to claim 49, having a polydespersity index of about 0.27 or below.

51. Cross-linked enzyme aggregate (CLEA) obtainable by the method according to claim 33, having a polydespersity index of about 0.27 or below.

52. A method for the preparation of a solid support comprising at least one immobilized enzyme, the method comprises the steps of a) obtaining at least one (such as a plurality of) CLEA according to the method according to claim 33, and b) immobilizing the CLEA(s) on or within a solid support.

53. A method for the preparation of a microfluidic device comprising at least one immobilized enzyme, the method comprises the steps of a) obtaining at least one (such as a plurality of) CLEA according to the method according to claim 33, and b) immobilizing the CLEA(s) on or within a surface of a microfluidic device.

54. A method for the preparation of a membrane microreactor comprising at least one immobilized enzyme, the method comprises the steps of a) obtaining at least one (such as a plurality of) CLEA according to the method according to claim 33, and b) immobilizing the CLEA(s) on the membrane surface of a membrane microreactor.

55. A solid support characterized in that at least one (such as a plurality of) CLEA according to claim 49 is (are) immobilized on or within.

56. A membrane microreactor characterized in that at least one (such as a plurality of) CLEA according to claim 49 is (are) immobilized on the membrane surface of the membrane microreactor.

57. A microfluidic system comprising a first reaction region, such as a first reaction vessel, a second reaction region, such as a second reaction vessel, which is in fluidic connection with the first reaction region, and an immobilization region, which is in fluidic connection with the second reaction region, wherein at least one (such as a plurality of) cross-linked enzyme aggregate (CLEA) as defined in claim 49 is (are) immobilized on or within the immobilization region.

58. The microfluidic system according to claim 57, wherein said immobilization region is selected from the group consisting of membrane microreactors, hollow fiber bioreactors, packed bed reactors, monolithic columns, microchannels and microchambers.

59. The microfluidic system according to claim 57, wherein said immobilization region is a membrane microreactor.

60. A process for producing a chiral amine comprising the step of catalyzing a transamination reaction using an amine transaminase cross-linked enzyme aggregate (ATA-CLEA) on a ketone compound and an amino donor, wherein the ATA-CLEA is an ATA-CLEA obtainable by the method as defined in claim 48.
